(19) United States
(12) Patent Application Publication
(10) Pub. No.: US 2015/0111273 A1
(43) Pub. Date: Apr. 23, 2015

(54) BIOHYDROGEN PRODUCTION METHOD AND REACTOR

(71) Applicant: GreenField Specialty Alcohols Inc., Toronto (CA)

(72) Inventor: Hisham Mohamed HAFEZ, London (CA)

(21) Appl. No.: 14/518,307

(22) Filed: Oct. 20, 2014

Related U.S. Application Data
(60) Provisional application No. 61/893,447, filed on Oct. 21, 2013.

(51) Int. Cl. C12P 3/00; C12M 1/107

(52) U.S. Cl. CPC C12P 3/00; C12M 21/04; C12M 23/36
USPC 435/168; 435/300.1

(57) ABSTRACT

A method for producing H₂, VFAs and alcohols from organic material is disclosed, including the steps of introducing organic material and microorganisms into a completely mixed bioreactor for producing H₂, CO₂, VFAs, and alcohols; sequestering CO₂ in the headspace of the reactor; recovering H₂ from the headspace; and recovering a first liquid effluent including microorganisms, VFAs, and alcohols. Also disclosed is a system for producing H₂, VFAs and alcohols from organic material, including a completely mixed bioreactor for dark fermentation; an input for supplying microorganisms and the organic material to be broken down; a CO₂ trap in the headspace and including a solid hydroxide for sequestration of the CO₂ gas from the headspace; and a gas output for removal of a gas effluent including H₂ gas from the headspace. The system and method provide higher H₂ production rates and a H₂ stream is substantially free of CO₂.

### CROSS-REFERENCE TO RELATED APPLICATIONS

This application claims the benefit of priority of U.S. Provisional Patent Application No. 61/893,447, entitled “BIOHYDROGEN PRODUCTION METHOD AND REACTOR” filed Oct. 21, 2013, which is hereby incorporated by reference in its entirety.

### FIELD

The present disclosure relates to the production of hydrogen, more particularly, the treatment of organic material with microorganisms for the production of hydrogen by dark fermentation.

### BACKGROUND

The problems of soaring energy demand and environmental pollution are addressed by various biological processes for the treatment of industrial wastes. Biohydrogen production through dark fermentation is one known process for the treatment of industrial waste and production of hydrogen.

Microorganisms are capable of producing hydrogen via either photosynthesis or preferably through fermentation [Matsunaga, T., Hatano, T., Yamada, A., Matsumoto, M., (2000) Microaerobic hydrogen production by photosynthetic bacteria in a double phase photobioreactor. Biotechnol. Bioeng. 68 (6), 647-651]. Organic pollutants are anaerobically converted to methane in two distinct stages: acidification and methanogenesis. Acidification produces hydrogen as a by-product which in turn is used as an electron donor by many methanogens at the second stage of the process [Fang, H. H. P. and Liu, H. (2002) Effect of pH on hydrogen production from glucose by a mixed culture. Bioresource Technology 82, 87-93]. Separation of the two stages is feasible for hydrogen collection from the first stage. The second stage is further used for treatment of the remaining acidification products, which includes mainly volatile fatty acids (VFAs).

The continuously stirred tank reactor (CSTR) has been the most widely used system for continuous hydrogen production [Li, C., Fang, H. H. P., (2007) Fermentative hydrogen production from wastewater and solid wastes by mixed cultures. Critical reviews in Env. Sci. and Tech., 37, 1-39]. Since in a CSTR biomass solids residence time (SRT) is the same as the hydraulic retention time (HRT), its concentration in the mixed liquor is highly affected by the recommended HRT of 1-12 h which is optimal for high hydrogen production rates [Li and Fang, 2007]. The maximum specific growth rate (μmax) for mixed culture of 0.333 h⁻¹ corresponds to an SRTₘᵢₙ of 3.0 h [Horiuchi J. I., Shimizu T., Tada K., Kanno T., Kobayashi M., (2002) Selective production of organic acids in anaerobic acid reactor by pH control. Bioresource Technol 82, 209-13].

Dark fermentative hydrogen (H₂) production is now being widely investigated for its promising advantages for the future of H₂ energy. It is a light-independent anaerobic process that utilizes a wide variety of feedstocks, and that can produce valuable metabolites such as acetic and butyric acids as by products [Nuri Azbar, David Levin (2012), State of the art and Progress in Production of Biohydrogen. Bentham Science Publishers]. However, dark fermentative H₂ production by thermodynamically favourable pathways is characterized by relatively low yields, with higher yields only possible through thermodynamically unfavourable pathways, thus requiring energy. In addition, the product gas mixture contains carbon dioxide (CO₂) which has to be separated [Azbar and Levin, 2012], since CO₂ is a major contaminant, specifically in fuel cell technologies that generate electricity from H₂ gas [D. C. Dayton (2001), Fuel Cell Integration—A Study of the Impacts of Gas Quality and Impurities. National Renewable Energy Laboratory], because proton exchange membrane fuel cells (PEMFCs) require high-purity H₂ (greater than 99%) [Larminie J, Dicks A (2000), Fuel cell systems explained. New York: Wiley].

The two most common pathways for dark fermentative H₂ production from glucose are the acetate and butyrate pathways (Equations 1 and 2), which limit the theoretical H₂ yield to between 2 and 4 moles of H₂ per mole of glucose. Both reactions are thermodynamically favourable (i.e. negative ΔG values) and the higher the acetate to butyrate ratio, the higher the H₂ yield. Thus, controlling the metabolism of the culture towards acetate formation is a key factor to achieve high H₂ yields [Sompong O-Thong, Poonsuk Prasertsan, Nils-Kare Birkeland (2009), Evaluation of methods for preparing hydrogen-producing seed inocula under thermophilic condition by process performance and microbial community analysis. Bioresource Technology 2009; 100: 909-918]. Also, in order to maximize H₂ yield, the metabolism should be directed away from alcohols (ethanol, butanol) and reduced acids (lactate) towards volatile fatty acids (VFA) production [David B. Levin, Lawrence Pitt, Murray Love (2004), Biohydrogen production: prospects and limitations to practical application. International Journal of Hydrogen Energy 2004; 29: 173-185]. However, propionate production decreases the H₂ yield since it is a H₂ consuming pathway (Equation 3).

C₆H₁₂O₆+2H₂O→2CH₃COOH+2CO₂+4H₂ ΔG_{R}°=−196.4 KJ (1)

C₆H₁₂O₆→CH₃(CH₂)₂COOH+2CO₂+2H₂ ΔG_{R}°=−224.2 KJ (2)

C₆H₁₂O₆+2H₂→2CH₃CH₂COOH+2H₂O ΔG_{R}°=−279.3 KJ (3)

The Le Chatelier principle states that a reversible reaction will shift to the right if one or more of its products are removed [Claire N. Sawyer, Perry L. McCarty, Gene F. Parkin (2003), Chemistry for Environmental Engineering and Science (5^{th} edition). McGraw-Hill Companies, Inc. 2003]. Therefore, removing CO₂ efficiently from the culture medium is expected to shift the H₂-producing pathways forward, increasing the H₂ production and preventing the consumption of Nicotinamide Adenine Dinucleotide (NADH) which is the base material for H₂ evolution [Kaushik Nath, Debabrata Das (2004), Improvement of fermentative hydrogen production: various approaches. Appl Microbiol Biotechnol 2004; 65: 520-529]. Kraemer and Bagley discussed several methods for improving the H₂ yield, one of which was removing dissolved H₂ and CO₂ from the liquid phase of the fermentation process [Jeremy T. Kraemer, David M. Bagley (2007), Improving the yield from fermentative hydrogen production. Biotechnol Left 2007; 29: 685-695].

One of the common techniques used for dissolved gas removal is gas sparging. Sparging is a technique which generally involves bubbling a chemically inert gas through a liquid to remove dissolved gas(es). Hussy et al. observed an increase in the H₂ yield from 1.0 to 1.9 mol/mol hexose converted using sucrose as the substrate in a CSTR operated at an HRT of 15 hours and achieving 95% sucrose conversion after sparging nitrogen (N₂) gas continuously in the reactor [I. Hussy, F. R. Hawkes, R. Dinsdale, D. L. Hawkes (2005), Continuous fermentative hydrogen production from sucrose and sugarbeet. International Journal of Hydrogen Energy 2005; 30: 471-483]. Kim et al. tested the utilization of N₂ as a sparging gas in H₂ production from sucrose in a CSTR operated at an HRT of 12 hours and loading of 40 gCOD/L·d and observed a 24% increase in the H₂ yield [Dong-Hoon Kim, Sun-Kee Han, Sang-Hyoun Kim, Hang-Sik Shin (2006), Effect of gas sparging on continuous fermentative hydrogen production. International Journal of Hydrogen Energy 2006; 31: 2158-2169]. Tanisho et al. observed a 110% increase in the H₂ yield by continuous purging of argon gas in a H₂ producing batch experiment by Enterobacter aerogenes using molasses as the carbon source. However, sparging processes require high capital cost processing equipment and maintenance.

Non-sparging techniques to decrease the dissolved gas concentrations can be increased stirring speed, applying vacuum in the headspace (i.e. decreasing the reactor headspace pressure), and using an immersed membrane to remove the dissolved gases [Kraemer and Bagley, supra]. Mandal et al., [supra] observed an increase of 105% in the H₂ yield of a batch H₂ producing experiment from glucose by Enterobacter cloacae by decreasing the headspace total pressure. The increase in H₂ yield was attributed to inhibition of H₂ consumption due to the decrease in total pressure that lead to the production of reduced by-products such as ethanol and organic acids [Mandal et al., supra]. By reducing H₂ and CO₂ contents, inhibition of homoacetogenesis was postulated to occur, preventing the consumption of H₂ and CO₂ to form acetate.

Jackson and McInerney stated that the degradation of a substrate is made thermodynamically possible through the removal of end products [Bradley E. Jackson, Michael J. McInerney (2002), Anaerobic microbial metabolism can proceed close to thermodynamic limits. Nature 2002; 415: 454-456]. Accordingly, glucose degradation through two pathways that are thermodynamically unfavourable could be shifted forward if CO₂ was removed from the headspace. Equations 4 and 5 show the two pathways that consume butyrate and propionate to produce acetate and H2.

CH₃(CH₂)₂COOH+2H₂O→2CH₃COOH+2H₂ ΔG_{R}°=+27.8 KJ (4)

CH₃CH₂COOH+2H₂O→CH₃COOH+CO₂+3H₂ ΔG_{R}°=+41.5 KJ (5)

Park et al. teach a batch process for producing H2 from glucose with initial sparging of the reactor to ensure anaerobic conditions, combined with CO₂ sequestration from the headspace, using a 30 wt % KOH solution [Wooshin Park, Seung H. Hyun, Sang-Eun Oh, Bruce E. Logan, In S. Kim (2005), Removal of headspace biological hydrogen production. Environ Sci Technol 2005; 39: 4416-4420]. However, they were able to reach a H₂ content of only 87.4% in the gas effluent. The incomplete CO₂ removal was due to the remaining CO₂ concentration in the liquid phase and some remaining N₂ gas from the initial sparging. Park et al. state that CO₂ removal did not substantially affect the concentrations of the other volatile acids and solvents. More importantly, Park et al. teach a batch process and acknowledge that the batch process results are not transferrable onto a continuous process. As an art skilled person will appreciate, continuous flow systems are fundamentally different from batch systems and batch process conditions can never be used in continuous systems for the same purpose or to achieve the same result. Continuous hydrogen production differs from batch production with respect to many important parameters i.e. hydraulic retention time (8 hours in continuous flow systems Vs. 2-5 days in batch systems), organic loading rate (only in continuously fed systems), pH (can be maintained constant in continuous flow, while in batches changes with time), concentration of biomass and the ratio of substrate to biomass (food-to-microorganisms ratio F/M) which is constant in continuously fed systems while decreases with time in batches due to the consumption of substrate. Park et al. definitely showed that CO₂ sequestration from the headspace improves H₂ yield in a batch system. However, they also stated that it was unclear whether the same approach would work in a continuous system and that more research was needed to find out if it worked at all. Specifically, Park et al. clearly stated that the conditions from the disclosed batch tests cannot necessarily be applied equally to a continuous system, particularly under conditions that affect the hydrogen production rate, such as different organic loadings and reactor retention times.

Liang et al. [Teh-Ming Liang, Sheng-Shung Cheng, Kung-Long Wu (2002), Behavioural study on hydrogen fermentation reactor installed with silicone rubber membrane. International Journal of Hydrogen Energy 2002; 27: 1157-1165] used a silicone rubber membrane to separate biogas from the liquid phase in a H₂ fermentation batch reactor using glucose as the substrate. The authors observed 15% and 10% increases in H₂ yield and H₂ production rate, respectively; however, they did not measure the VFAs concentrations.

Mandal et al. [2006] suggest the removal of both H₂ and CO₂ from the headspace by vacuum, to reduce acetate production. Mandal et al. neither suggest any selective removal of only CO₂ nor the use of sequestration for CO₂ removal. The Mandal et al. study focused on lowering the hydrogen partial pressure in batches of hydrogen production by applying a negative pressure to the gas collector, which is connected to the headspace of the reactor. The removal of carbon dioxide in this study is primarily due to the vacuum (negative) pressure being applied. The use of KOH in the gas collector had no influence on the reactor kinetics. Their experiment was based on Le Chatelier's principle that removing both gaseous products by lowering the total pressure would shift the reaction forward.

The problem with the aforementioned techniques for dissolved gas removal from the liquid phase is that the effluent gas is a mixture of gases that should be separated in order to benefit from each separately. Moreover, since the major problem with H₂ utilization in fuel cells is contamination with CO₂, a process is desired which provides for reliable CO₂ removal from biohydrogen, preferably a process which combines CO₂ removal with improved H₂ yields.

### SUMMARY OF THE INVENTION

It is an object of the present disclosure to obviate or mitigate at least one disadvantage of previous methods and systems for the production of hydrogen from organic material.

The inventor of the present application has now discovered a process for dark fermentative H₂ production, which includes continuous CO₂ sequestration within the reactor headspace for producing a substantially CO₂ free H₂ stream. The inventor surprisingly discovered that by performing CO₂ capture directly within the headspace of a continuous reactor, the amount of CO₂ sequestered can be increased to 100% of the CO₂ produced in the reactor. By using sequestration of the CO₂ gas, which means the capture of the CO₂ gas in the headspace and conversion of the CO₂ gas in the headspace into a non-gaseous, solid form, the bicarbonate, it is possible to influence the reactor kinetics without physically removing the CO₂ gas from the reactor itself. Moreover, by capturing the CO₂ gas and converting it to bicarbonate in the headspace of the reactor, the volume of CO₂ based reaction product to be handled is significantly reduced. More importantly, by sequestering the CO₂ gas in the headspace, the CO₂ gas is completely removed from the reactor kinetics with the added side effect that the H₂ production rate is increased. The CO₂ gas is also substantially completely removed from the reactor headspace, with the further side effect of the H₂ gas in the headspace being substantially free of CO₂. Thus, the process of the invention not only provides significantly improved H₂ yields previously not attainable, but at the same time provides a virtually CO₂ free H₂ stream directly from the reactor, obviating any further separation of the CO₂ and H₂ gases produced in the reactor or cleaning of the H₂ gas downstream of the reactor. This significantly reduces capital cost and makes the H₂ gas production more economical. It further allows for the separate removal of H₂ and CO₂ directly from the reactor without any further separation steps.

In one preferred embodiment, the present method for producing hydrogen by dark fermentation, from organic material, comprises the steps of
introducing organic material and microorganisms into a completely mixed bioreactor for breaking down the organic material into products including H₂ gas, CO₂ gas, volatile fatty acids, and alcohols by dark fermentation;
continuously sequestering CO₂ gas within a headspace of the bioreactor for capturing the CO₂ as bicarbonate within the headspace; and
continuously or discontinuously recovering at least a portion of the H₂ gas from the headspace under vacuum, whereby the recovered H₂ gas is substantially free of CO₂.

In another embodiment, the step of sequestering CO₂ within the headspace includes the further step of discontinuously removing at least a portion of the bicarbonate from the headspace.

In still another embodiment, the step of sequestering CO₂ includes continuously maintaining a metal hydroxide in the headspace for continuously capturing the gaseous CO₂ as metal bicarbonate within the headspace, thereby removing the CO₂ gas from the headspace. The metal hydroxide is preferably used in solid form.

Preferably, the metal hydroxide is an alkali metal hydroxide, more preferably KOH or NaOH, most preferably 100% pure KOH or NaOH pellets.

In a further embodiment, the method includes the further step of maintaining a concentration of microorganisms in the completely mixed bioreactor at a preselected value.

In still a further embodiment, the method includes the further step of controlling the pH of the completely mixed bioreactor. Preferably, the pH of the completely mixed bioreactor is maintained within a range of 3 to 6.8, most preferably at about 5.2.

Microorganisms useful in the present invention include one or more of the species selected from the group consisting of Clostridium species, such as C. butyricum, C. beijerinckii, C. acetobutyricum and C. bifermentants, Enterobacter species, such as Enterobacter aerogenes, Bacillus species such as B. megaterium, B. thuringiensis, and Rhodobacter species, such as R. sphaeroides.

Preferably, the completely mixed bioreactor is a reactor selected from the group consisting of a single continuously stirred tank reactor, a multi-stage continuously stirred tank reactor, an up-flow anaerobic sludge blanket reactor, an expanded bed granular sludge blanket reactor, a down-flow anaerobic granular media reactor, an up-flow anaerobic granular media reactor, an anaerobic baffled tank reactor, an anaerobic migrating blanket reactor, and an anaerobic fluidized bed bioreactor.

The method disclosed herein may be implemented through an integrated biohydrogen reactor clarifier system (IBRCSs) including a CSTR, followed by a gravity settler for acetone-butanol-ethanol (ABE) fermentation of organic material. The ABE fermentation results in products including for example acetone, butanol, ethanol, acetic acid, butyric acid, hydrogen gas, and/or carbon dioxide. Hydrogen gas and carbon dioxide are recovered separately from the CSTR. The biomass concentration in the CSTR reactor is kept at the desired range through biomass recirculation from the bottom of the gravity settler and/or biomass wastage from the gravity settler's underflow. A separation process is used to separate further biomass from the acetone, butanol, ethanol, acetic acid, butyric acid, etc., which are recovered. The biomass is provided to a biomethane generator, also referred to as biomethanator, for the production of methane gas.

In yet a further embodiment, the present description provides a system for producing hydrogen, methane, volatile fatty acids, and alcohols from organic material, comprises:
a completely mixed bioreactor for dark fermentation;
an input for supplying to the bioreactor microorganisms and the organic material to be broken down by the microorganisms into products including H₂ gas, CO₂ gas, volatile fatty acids (VFA) and alcohols;
a CO₂ trap in a headspace of the reactor, the CO₂ trap including a solid hydroxide for continuous or discontinuous sequestration of the CO₂ gas from the headspace and capture of the CO₂ as bicarbonate within the headspace;
a gas output for removal of a gas effluent including H₂ gas from the headspace; and
a liquid output for removing a first liquid effluent including at least a portion of the microorganisms, the volatile fatty acids, and the alcohols from the bioreactor.

In another embodiment, the CO₂ trap includes a solid metal hydroxide, preferably an alkali metal hydroxide, more preferably KOH or NaOH, most preferably 100% KOH or NaOH pellets.

In a further embodiment, the system comprises two or more CO₂ traps separately removable from the headspace for removal of the CO₂ captured as bicarbonate during continuous operation of the reactor.

In still another embodiment, the system further comprises a gravity settler in fluid communication with the liquid output for separating the first liquid effluent into a settled out first biomass including at least a portion of the microorganisms and a second liquid effluent including at least a portion of the volatile fatty acids, the alcohols and the microorganisms; and means for feeding the first biomass from the gravity settler to the completely mixed bioreactor for maintaining a concentration of microorganisms in the completely mixed bioreactor at a preselected value.

In another embodiment, the system further comprises a dispenser for dispensing chemicals for pH adjustment into the completely mixed bioreactor.

Moreover, the system preferably includes a temperature controller for controlling a temperature of the bioreactor.

The completely mixed bioreactor is preferably a reactor selected from the group consisting of a single continuously stirred tank reactor, a multi-stage continuously stirred tank reactor, an up-flow anaerobic sludge blanket reactor, an expanded bed granular sludge blanket reactor, a down-flow anaerobic granular media reactor, an up-flow anaerobic granular media reactor, an anaerobic baffled tank reactor, an anaerobic migrating blanket reactor, and an anaerobic fluidized bed bioreactor.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 is a flow diagram of a process for producing hydrogen gas, carbon dioxide, volatile fatty acids, and alcohols from organic biomass;

FIG. 2 is a schematic of a system for producing hydrogen gas, carbon dioxide, volatile fatty acids and alcohols from organic material;

FIG. 3 illustrates the hydrogen content with and without CO₂ sequestration;

FIG. 4 illustrates hydrogen production rates with and without CO₂ sequestration; and

FIG. 5 illustrates hydrogen production yield with and without CO₂ sequestration.

### DETAILED DESCRIPTION OF EXEMPLARY EMBODIMENTS

Generally, the present disclosure provides a method and integrated system for the production of biohydrogen by dark fermentation and preferably other chemicals such as bicarbonate, ethanol, butanol, acetic acid, propionic acid, and butyric acid from organic material, preferably in a continuously stirred reactor (CSTR). A downstream gravity settler may be integrated into the system after the CSTR. Embodiments of the method and system are disclosed herein. However, the disclosed embodiments are merely exemplary, and the method and system may be embodied in many various and alternative forms.

As used herein, the terms “about” and “approximately” are used in conjunction with ranges of dimensions, concentrations, temperatures, or other physical or chemical properties and characteristics. Use of these terms is meant to cover slight variations that may exist in the upper and lower limits of the ranges of properties and characteristics.

As used herein, the term “completely mixed bioreactor” means a vessel including a mechanism for agitating the contents of the vessel (e.g. by hydraulic agitation, mechanical agitation, etc.) for use with microorganisms in suspension and a growth media, (e.g. a growth media comprised of nutrients such as organic carbon, nitrogen-containing compounds, phosphorous-containing compounds, and trace mineral solutions, etc.). A continuously stirred reactor (CSTR) is an example of a completely mixed bioreactor.

As used herein, the term “microorganisms” means microorganisms capable of fermenting organic material under anaerobic (not microaerobic) conditions to produce hydrogen or methane, carbon dioxide, and a variety of organic acids and alcohols. Species of microorganisms within this term may include, for example, one or combination of various Clostridium species such as C. butyricum, C. beijerinckii, C. acetobutyricum and C. bifermentants, Enterobacter species such as Enterobacter aerogenes, Bacillus species such as megaterium, thuringiensis, and other anaerobic bacteria (e.g. Rhodobacter sphaeroides).

As used herein, the term “organic material” refers to material including carbon and hydrogen in its molecular structure, for example alcohols, ketones, aldehydes, fatty acids, esters, carboxylic acids, ethers, carbohydrates, proteins, lipids, polysaccharides, monosaccharide, cellulose, nucleic acids, etc. Organic material may be present for example, in waste (e.g. industrial waste streams), organic fluid streams, biomass, etc.

### Process

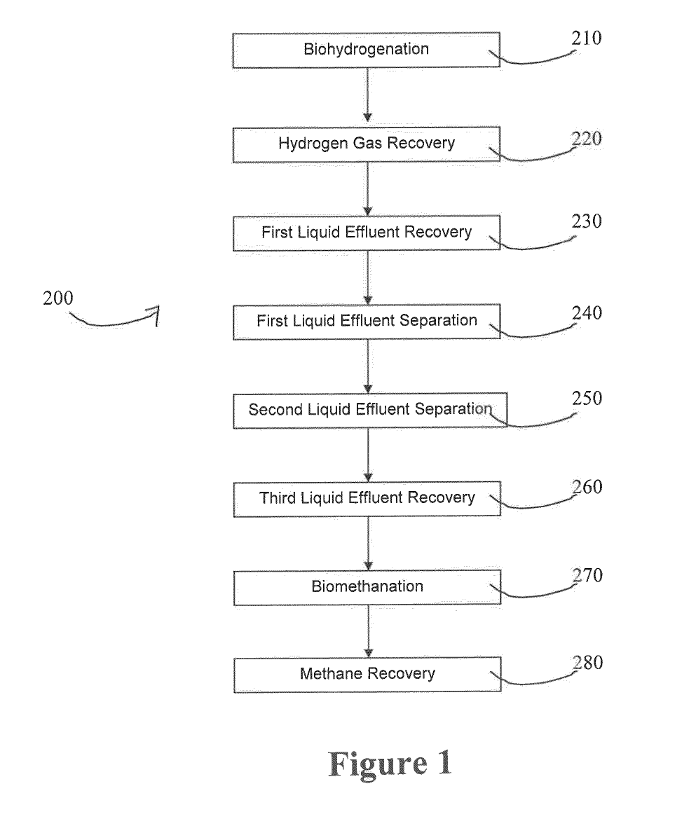

FIG. 1 is a flow diagram of a process 200 for producing hydrogen gas, carbon dioxide, volatile fatty acids, and alcohols from organic biomass. The process 200 includes a biohydrogeneration step 210, a CO₂ sequestration step 215, a hydrogen gas recovery step 220, a first liquid effluent recovery step 230, and a first liquid effluent separation step 240. In a variant of the process, which results in the production of methane and CO₂, the process further includes a second liquid effluent separation step 250, a third liquid effluent recovery step 260, a biomethane generation step 270, also referred to as biomethanation step 270, and a methane recovery step 280. The steps 210, 220, 230, 240, 250, 260, 270, 280 may be carried out in a continuous fashion where some or all of the steps 210, 220, 230, 240, 250, 260, 270, 280 are being performed simultaneously and continuously or discontinuously, in contrast with a batch approach where the steps 210, 220, 230, 240, 250, 260, 270, 280 would be carried out sequentially rather than simultaneously.

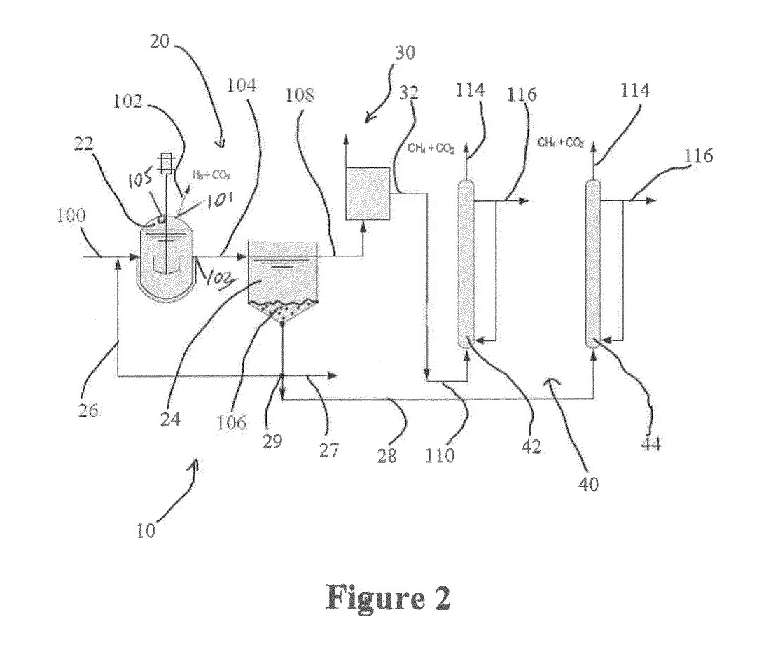

In the biohydrogeneration step 210, organic material and microorganisms are provided into a completely mixed bioreactor (e.g. the completely mixed bioreactor 22 of FIG. 2) for breaking down the organic material into products including H₂, CO₂, volatile fatty acids, and alcohols. In the CO₂ sequestration step, CO₂ gas is captured in a headspace of the bioreactor and converted it into bicarbonate within the headspace. By sequestering the CO₂ gas in the headspace, the CO₂ gas is effectively removed from the reactor kinetics without physical removal of the CO₂ from the reactor. In the hydrogen gas recovery step 220, at least a portion of the H₂ gas is recovered from the completely mixed bioreactor under vacuum. In the first liquid effluent recovery step 230, at least a portion of a first liquid effluent is recovered from the completely mixed bioreactor, the first liquid effluent including at least a portion of the microorganisms, the volatile fatty acids, and the alcohols.

In the CO₂ sequestration step, the bicarbonate is collected in the headspace and discontinuously removed from the headspace. In the CO₂ sequestration step CO₂ gas is captured and removed from the reactor kinetics by reaction with a solid hydroxide, preferably a metal hydroxide, more preferably an alkali metal hydroxide, most preferably KOH or NaOH. The metal hydroxide is preferably in the form of 100% KOH or NaOH pellets. Using CO₂ gas sequestration in the headspace has multiple advantages. CO₂ sequestration within the reactor headspace produces a substantially CO₂ gas free H₂ stream. It was surprising to the inventor that by performing CO₂ gas capture directly within the reactor headspace, the amount of CO₂ gas captured can be raised to 100% of the CO₂ produced in the reactor. Moreover, continuously completely removing the CO₂ gas from the headspace by CO₂ sequestration has the further side effect that the H₂ production is increased. This is likely due to the complete suppression of propionate formation, which was also surprisingly observed. Thus, the process of the invention not only provides significantly improved H2 yields previously not attainable, but at the same time results in a virtually CO₂ free H₂ stream directly from the reactor, obviating any further separation of the CO₂ and H₂ gases downstream from the reactor. Compared to the known methods of using a KOH solution to react with gaseous H₂/CO₂ in a vessel separate from the reactor, the present system requires less energy and equipment since the gas does not have to be transferred from the reactor through the KOH solution using some type of mechanical device such as a blower. This significantly reduces capital cost and makes H₂ gas production more economical. It further allows for the separate removal of H₂ and CO₂ from the reactor.

In addition to the lower capital and operating costs associated with the present solid/gas sequestration reaction system, the amount of CO₂ sequestered increases significantly from 79% (Mandel et al. sequestered 19.3% from original 24.5%) to about 100%.

In the first liquid effluent separation step 240, at least a portion of the first liquid effluent is fed into a gravity settler (e.g. the gravity settler 24 of FIG. 2) for separating at least a portion of the first liquid effluent into a first biomass including at least a portion of the microorganisms and a second liquid effluent including at least a portion of the volatile fatty acids, the alcohols and the microorganisms. Although other separators, such as membrane separators are known, they are capital intensive and much harder to operate. In the second liquid effluent separation step 250, at least a portion of the second liquid effluent is fed to a separation module (e.g. the separation module 30 of FIG. 2) for separating at least a portion of the second liquid effluent into a second biomass including at least a portion of the microorganisms and a third liquid effluent including at least a portion of the volatile fatty acids and the alcohols. At least a portion of the third liquid effluent is recovered in the third liquid effluent recovery step 260.

The first liquid effluent separation step 240 may include recirculating at least a portion of the first biomass to the completely mixed bioreactor to maintain a concentration of microorganisms in the completely mixed reactor at a preselected value.

In the biomethanation step 270, at least a portion of the first biomass, the second biomass, or both, is recovered and provided to a biomethanator (e.g. the biomethanator 40 of FIG. 2) for producing CH₄ and CO₂. The terms biomethane generator and biomethanator are used interchangeably in this description and are both intended to refer to reactors for biological production of methane. At least a portion of the CH₄ and CO₂ is recovered in the methane recovery step 280.

The second liquid effluent separation step 250 may include the application of a variety of separation processes, for example membrane solvent separation.

The pH range may be controlled in the completely mixed bioreactor during the biohydrogeneration step 210. For example, the pH range may be kept within a range of 3 to 6.8 depending on the desired end products. Preferably, the pH is maintained at about 5.2 to maximize the H₂ production rate.

The pH range may be controlled in the biomethanator during the biomethanation step 270. The temperature may be controlled in the completely mixed bioreactor during the biohydrogeneration step 210. For example, the temperature may be kept within a range of about 25° C. to about 37° C.

The temperature may be controlled in the biomethanator during the biomethanation step 270. For example, the temperature may be kept within a range of about 25° C. to about 37° C.

The microorganisms useful for application in the system of the present application include Clostridium species, such as C. butyricum, C. beijerinckii, C. acetobutyricum and C. bifermentants, Enterobacter species, such as Enterobacter aerogenes, Bacillus species such as B. megaterium, B. thuringiensis, and Rhodobacter species, such as R. sphaeroides.

### System

FIG. 2 is a schematic of a system 10 for producing hydrogen gas, carbon dioxide, methane, volatile fatty acids, and alcohols from organic material. Further products produced by the system 10 may include acetone, ethanol, butanol, acetic acid, propionic acid, and butyric acid. The system 10 includes a biohydrogenerator 20, a separation module 30, and a biomethane generator or biomethanator 40.

The biohydrogenerator 20 includes a completely mixed bioreactor 22 having an inlet for receiving organic material 100 into the completely mixed bioreactor 22. Microorganisms are added to the completely mixed bioreactor 22 to break down the organic material 100, producing H₂ and CO₂. The reactor 22 further includes a gas outlet 101 for H₂ gas 102 and a liquid outlet 103 for a first liquid effluent 104. The first liquid effluent 104 may include, for example, microorganisms, volatile fatty acids (e.g. acetic acid, butyric acid, etc.), alcohols (e.g. ethanol, butanol, etc.), acetone, etc. A CO₂ trap 105 is included in the headspace of the bioreactor 22, which trap includes a hydroxide in solid form, preferably an alkali metal hydroxide such as KOH or NaOH, most preferably 100% KOH or NaOH pellets. The CO₂ trap 105 is preferably removable from the bioreactor during operation of the biohydrogeneration. Most preferably, the bioreactor 22 includes 2 or more CO₂ traps, which can be individually and independently removed from the bioreactor and replaced to allow for continuous CO₂ sequestration even during replacement of one of the CO₂ traps.

The biohydrogenerator 20 further includes a gravity settler 24 downstream of the completely mixed bioreactor 22 and in fluid communication with the completely mixed bioreactor 22 for receiving the first liquid effluent 104 from the completely mixed bioreactor 22. In the gravity settler 24, the first liquid effluent 104 settles into a first biomass 106 and a second liquid effluent 108. The second liquid effluent 108 may include, for example, microorganisms, volatile fatty acids (e.g. acetic acid, propionic acid, butyric acid, etc.), alcohols (e.g. ethanol, butanol, etc.), acetone, etc.

A recirculation conduit 26 provides fluid communication from the bottom of the gravity settler 24 to the completely mixed bioreactor 22 for recirculating the first biomass 106 from the gravity settler 24 to the completely mixed bioreactor 22. An output conduit 27 from the bottom of the gravity settler 24 is for discharging and disposal the first biomass 106. A first biomethanator conduit 28 provides fluid communication from the bottom of the gravity settler to the biomethanator 40 for circulating the first biomass 106 from the gravity settler 24 to the biomethanator 40. A valve 29 allows selection of flow through one or more of the recirculation conduit 26, the output conduit 27, and the first biomethanator conduit 28.

The separation module 30 is in fluid communication with the gravity settler 24 for receiving the second liquid effluent 108. In the separation module 30, the second liquid effluent 108 may be separated into a second biomass 110 and a third liquid effluent 112 by application of a separation process. The third liquid effluent 112 may include, for example, volatile fatty acids (e.g. acetic acid, propionic acid, butyric acid, etc.), alcohols (e.g. ethanol, butanol, etc.), acetone, etc. A second biomethanator conduit 32 provides fluid communication from the separation module 30 to the biomethanator 40 for circulating the second biomass 110 from the separation module 30 to the biomethanator 40.

The biomethanator 40 is downstream of, and in fluid communication with, the gravity settler 24, the separation module 30, or both. The biomethanator 40 may receive biomass from the biohydrogenerator 20, the separation module 30, or both, for being broken down into CH₄ and CO₂ 114, and a liquid waste 116 containing residual organics and microorganisms.

The biomethanator 40 may include a first biomethanator vessel 42, a second biomethanator vessel 44, or both. The first biomethanator vessel 42 is in fluid communication with the first biomethanator conduit 28 for receiving the first biomass 106 from the gravity settler 24. The second biomethanator vessel 44 is in fluid communication with the second biomethanator conduit 32 for receiving the second biomass 110 from the separation module 30.

The system 10 may include a temperature controller (not shown) for controlling the temperature in the completely mixed bioreactor 22, in the biomethanator 40, or both. A typical temperature range in which the temperature of the contents of both the completely mixed bioreactor 22 and biomethanator 40 is maintained is between about 25° C. and about 37° C.

The system 10 may include a dispenser (not shown) for dispensing nutrients and pH adjustment compounds into the completely mixed bioreactor. The nutrients may include, for example, nitrogen containing compounds, phosphorous containing compounds, trace metals including iron, manganese, magnesium, calcium, cobalt, zinc, nickel, copper, etc. The pH adjustment compounds may include, for example, soda ash, sodium bicarbonate, sodium hydroxide, calcium hydroxide, magnesium hydroxide, nitric acid, hydrochloric acid, etc.

### Operation

The system 10 can be applied to practice an embodiment of the process 200. The organic material 100 enters the completely mixed bioreactor 22 and is broken down microbiologically by hydrogen producing microorganisms, resulting in products including the H₂ gas and CO₂ gas, and the first liquid effluent 104. The CO₂ gas is sequestered by the hydroxide in the CO₂ trap and captured as bicarbonate in the trap. A H₂ stream 102 substantially free of CO₂ is continuously removed from the completely mixed bioreactor 22. The first liquid effluent 104 flows to the gravity settler 24. The bicarbonate captured in the CO₂ trap remains in the CO₂ trap and is discontinuously removed from the bioreactor 22.

In the gravity settler 24, at least a portion of the microorganisms settle to the bottom of the gravity settler 24, resulting in the first biomass 106 and the second liquid effluent 108. The first biomass 106, in whole or in part, may be recirculated to the completely mixed bioreactor 22, provided to the biomethanator 40, disposed of, or any combination thereof. The second liquid effluent 108 flows into the separation module 30.

In the separation module 30, at least a portion of the second liquid effluent 108 settles into a second biomass 110 and a third liquid effluent 112. The third liquid effluent 112 is emitted from the separation module 30 and recovered. The second biomass 110 may be provided to the biomethanator 40. Providing the second biomass 110 to the completely mixed bioreactor is also possible, but not necessary in the presence of a recycle stream from the gravity settler 24.

The first biomass 106 is provided to the first biomethanator vessel 42 through the first biomethanator conduit 28. The second biomass 110 is provided to the second biomethanator vessel 44 through the second biomethanator conduit 34. In the biomethanator 40, the first biomass 106, the second biomass 110, or both, are broken down microbiologically, resulting in production of the CH₄ and CO₂ 114. The CH₄ and CO₂ 114 are emitted from the biomethanator 40 and recovered. The liquid waste 116 is discharged from the biomethanator 40, recirculated into the biomethanator 40, or both.

Exemplary operating conditions and system configurations will be discussed in the following for the purpose of example only and without limiting the scope of the invention to less than the subject matter defined in the claims.

### EXAMPLES

### IBRCS Setup

During testing of system 10, three major changes in the effluent volatile fatty acids (VFA) concentrations were observed with CO₂ sequestration, an increase in the acetate concentration by an average of 45%, a decrease in the butyrate concentration to an average of 51% of its original concentration, and a complete elimination of the propionate production. Moreover, during testing, the hydrogen production rates under two different organic loading rates were 63 L H2/d (at 9 g/L of glucose) and 132 LH2/d (at 17 g/L of glucose) and almost 100% pure hydrogen was achieved.

Two integrated biohydrogen reactor clarifier systems (IBRCSs) consisting of a CSTR (7 L working volume), followed by a gravity settler (8 L volume) were operated in parallel at two different OLRs. For further details on the system design, refer to Hafez et al. [2009]. OLR-1 and OLR-2 were 25.7 and 51.4 gCOD/L-d, respectively. A cylindrical CO₂ trap (0.25 L volume) with a porous bottom was introduced into the system and fixed in the reactor cover. Each OLR was operated in two conditions in series: 18 days without CO₂ sequestration, followed by 17 days with CO₂ sequestration through the addition of KOH pellets (60 g) in the CO₂ trap fixed in the headspace.

### Seed Sludge and Substrate

Anaerobic digester sludge (ADS) was collected from St. Mary's wastewater treatment plant (St. Mary's, Ontario, Canada) and preheated at 70° C. for 30 min to be used as the seed. Glucose was used as the substrate with two different concentrations of 8 g/L (OLR-1) and 16 g/L (OLR-2). The feed contained sufficient inorganics at the following concentrations (mg/L): CaCl₂, 140; MgCl₂.6H₂O, 160; MgSO₄.7H₂O, 160; Na₂CO₃, 200; KHCO₃, 200; K₂HPO₄, 15; urea, 1500; H₃PO₄, 845; and trace mineral solution with composition as follows (mg/L): FeCl₂.4H₂O, 2000; H₃BO₃, 50; ZnCl₂, 50; CuCl₂, 30; MnCl₂.4H₂O, 500; (NH₄)₆MO₇O₂₄, 50; CoCl₂.6H₂O, 50; NiCl₂, 50; ethylenediaminetetraacetate, 0.5; and concentrated HCl, 1170. Buffer used in the feed was NaHCO₃ at concentrations of 3 and 5 g/L for systems operating at OLR-1 and OLR-2, respectively. A pH of 5.2 was maintained during the experiment using NaHCO₃ solution with a concentration of 168 g/L.

### Analytical Methods

The volume of biogas produced was measured using a wet-tip gas meter (Rebel wet-tip gas meter company, Nashville, Tenn., USA), while the biogas composition was determined using a gas chromatograph (Model 310, SRI instruments, Torrance, Calif.) with a thermal conductivity detector (TCD) at a temperature of 90° C. and a molecular sieve column (Molesieve 5A, mesh 80/100, 6 ft*⅛ in) at a temperature of 105° C. Argon was used as the carrier gas at a flow rate of 30 mL/min. The volatile fatty acids (VFAs) concentrations were analyzed using a gas chromatograph (Varian 8500, Varian Inc., Toronto, Canada) with a flame ionization detector (FID) at a temperature of 250° C. equipped with a fused silica column (30 m*0.32 mm) at a temperature of 110° C. Helium was used as the carrier gas at a flow rate of 5 mL/min. The total and volatile suspended solids (TSS, VSS) were measured according to standard methods [APHA 1995]. Glucose was analyzed by Genzyme Diagnostics P.E.I. Inc. PE Canada glucose kit. HACH methods and testing kits (HACH Odyssey DR/2500) were used to measure the total and soluble chemical oxygen demands (TCOD, SCOD).

### Hydrogen Production

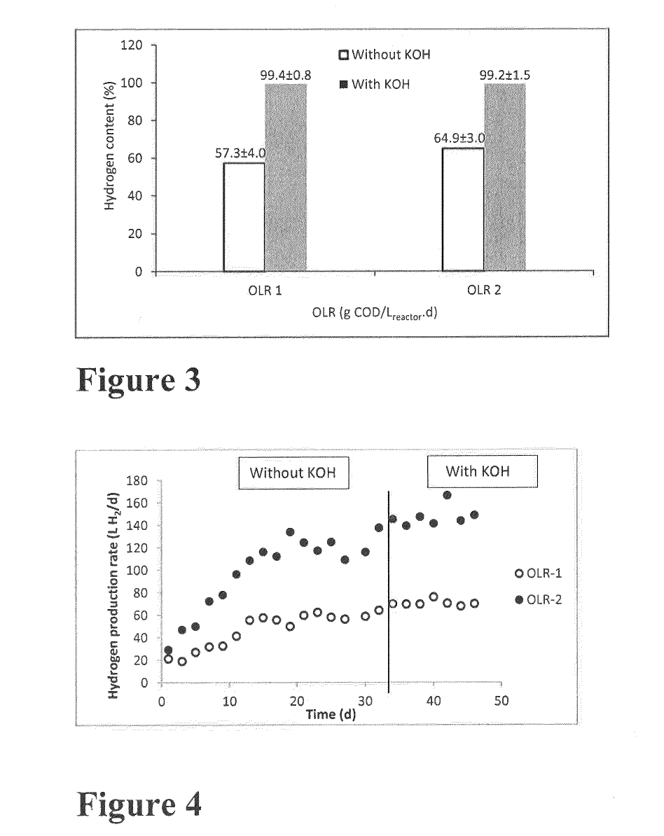

FIG. 3 shows the change in H₂ content due to the addition of KOH in the headspace. H₂ content reached 57.3±4% and 64.9±3% at OLR-1 and OLR-2, respectively without KOH, increasing rapidly to 100% in both cases after application of KOH in the headspace. Park et al. [2005] achieved only 87.4% H₂ after adding KOH in the headspace of H₂ producing batch experiments, due to incomplete sequestration of headspace CO₂. It must be asserted that the headspace biogas composition is dictated not only by the liquid phase CO₂ and H₂ production rates but also by the mass transfer from liquid to gas. Since in batches, after the maximum production rates are established, rates usually decline with time due to lower substrate utilization rates, the extrapolation of batch biogas composition data to continuous-flow systems depends on numerous factors related to operational conditions i.e. OLR, HRT, biomass concentration, etc.

H₂ production rates increased from 57 to 70 L H₂/d and from 118 to 146 L H₂/d, in both OLR-1 and OLR-2, respectively. FIG. 2 shows an average increase of 23.5% in the H₂ production rates, where after 12 days a steady state performance was reached, with an average fluctuation in production rates of 3.4% and 8.7% in both OLR-1 and OLR-2, respectively. H₂ production rates based on liters of reactor volume before applying KOH were 8.2±0.5 and 16.9±1.0 L/L−d, which are consistent with Hafez et al. [2010] who achieved 9.6 and 19.6 L/L−d. After applying KOH, the rates increased to 10±0.4 and 20.9±1.1 L/L−d for both OLR-1 and OLR-2, respectively. It is postulated that removing CO₂ from the headspace forced reactions 1, 2, and 3 to go forward, which lead to an increase in the H₂ production rate in order to compensate for the decrease in the CO₂ concentration. FIG. 3 illustrates the hydrogen content with and without CO₂ sequestration, while FIG. 4 illustrates hydrogen production rates with and without CO₂ sequestration.

### Hydrogen Yields

H₂ yields achieved at OLR-1 and OLR-2 before sequestering CO₂ were 2.42±0.15 and 2.50±0.18 mol/mol, respectively, which is consistent with Hafez et al. [2010] who achieved H₂ yields of 2.8 and 2.9 mol/mol at the same OLRs and HRT in the IBRCS. These results are 27% higher than the maximum H₂ yield of 1.93 mol/mol that was achieved by Zhang et al. [2006] at an OLR of 32.1 gCOD/L-d and an HRT of 8 hours in a continuous stirred tank reactor using glucose and mixed anaerobic culture.

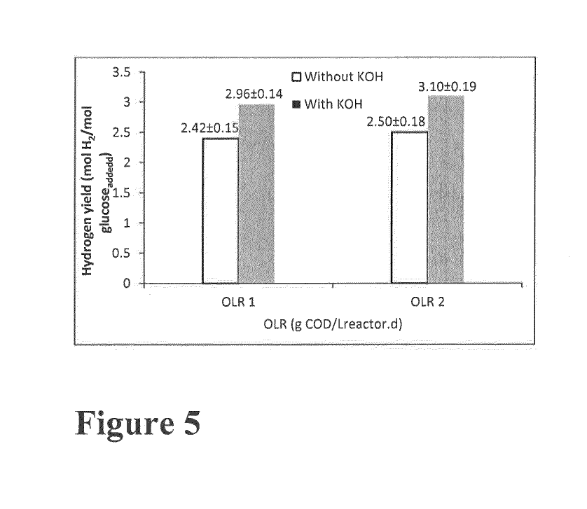

FIG. 5 shows the increase in H₂ yield due to headspace CO₂ sequestration. An average increase of 23% was achieved at both OLRs; with average yields of 2.96±0.14 and 3.10±0.19 mol/mol achieved at OLR-1 and OLR-2 with CO₂ sequestration. The increase in the H₂ yield is attributed to shifting reactions 1 and 2 forward due to CO₂ sequestration according to Le Chatelier principle [Sawyer et al., 2003]. However, only an increase of 23% was observed since H₂ yields using the IBRCS before applying CO₂ sequestration are already high (2.42±0.15 and 2.50±0.18 mol/mol). With a maximum theoretical H₂ yield of 4 mol/mol, maximum practical yield of 3.4 mol/mol taking the biomass yield into consideration, and maximum achieved yield of 3 mol/mol [Hafez et al., 2010], the 23% increase in the yield due to sequestering CO₂ achieved 91.2% of the practical yield. The impact of CO₂ sequestration on the H₂ yield would be more drastic at the low H₂ yields achieved by other systems using glucose as the substrate and anaerobic digested sludge as the seed, such as 1.8 mol/mol in a CSTR [Zhang et al., 2007; Show et al., 2007], 1.57 mol/mol in an agitated granular sludge bed reactor [Wu et al., 2008], and 1.83 mol/mol in an AFBR [Zhang et al., 2008; Show et al., 2010]. FIG. 5 illustrates hydrogen production yield with and without CO₂ sequestration.

### 3.3 Volatile Fatty Acids (VFA)

Table 1 shows the effluent VFA concentrations at OLR-1 and OLR-2 before and after applying KOH in the headspace. It is noteworthy that there were three major changes in the effluent VFA concentrations after sequestering CO₂; an increase in the acetate concentration by an average of 45%, a decrease in the butyrate concentration to an average of 51% of its original concentration, and a complete elimination of the propionate. On the contrary, Park et al. [2005] observed a decrease in the acetate concentration after applying KOH in the headspace of their batch experiments due to inhibition of homoacetogenesis, in addition to an increase in the ethanol production, with acetate and ethanol as the two main by-products. Also, Kim et al. [2006] observed a decrease in the acetate concentration to only 35% of its original value, and an increase in both butyrate and propionate concentrations by 101% and 28%, respectively, after applying continuous N₂ and CO₂ gas sparging in a CSTR producing H₂ from sucrose at an OLR of 40 gCOD/L·d and an HRT of 12 hours. However, the aforementioned authors observed low H₂ yields of 0.75, 0.93, and 1.20 mol/mol hexose added without gas sparging, with N₂ sparging, and with CO₂ sparging, respectively, indicative of H₂ production mainly through the butyrate pathway. It should be noted that since the aforementioned systems were operated at low biomass concentrations of −1000 mgVSS/L, specific H₂ production rates are lower than in this study. Interestingly with N₂ sparging only, Kim et al. [2006] observed a 24% increase in H₂ yield in agreement with the 24% observed in this study, without any changes in microbial communities i.e. the predominance of the butyrate pathway without gas sparging continued after N₂ sparging. The aforementioned authors repeated however that with CO₂ sparging, the improved yield is due to inhibition of acetogens and lactic acid bacteria, which compete with H₂ producers.

High H2 yields have been associated with acetate and butyrate as fermentation products [Hawkes et al., 2002]. Acetate and butyrate pathways limit the H₂ yield to the range of 2 to 4 moles of H₂ per 1 mole of glucose (Equation 1 and 2). On the other hand, low H₂ yields have been associated with propionate production [Hawkes et al., 2002]. The propionate pathway is a H2 consuming reaction which affects the yields negatively (Equation 3), so production of propionate should be avoided [Vavilin 1995]. In addition, from a thermodynamic point of view, Equation (5) shows a propionate consuming reaction that produces H₂ and acetate is thermodynamically unfavourable (positive AG). Consequently, removing CO₂ from the headspace will shift reaction (5) forward changing the reaction to be thermodynamically favourable. Accordingly, both H₂ and acetate production will increase, and propionate will be consumed, which explains the increase in acetate concentration and the withdrawal in propionate concentration. This pathway (Equation 5) will increase the range of theoretical H₂ production to reach 3 to 4 moles of H₂ per mole of glucose, with acetate as the main by-product.

Theoretical H₂ production from VFAs produced was calculated based on 0.84 L H₂/g acetate and 0.58 L H₂/g butyrate (Equations 1 and 2). The theoretical values shown in Table 1 were consistent with the H₂ measured during the experiment with an average theoretical to measured ratio of 116%.

TABLE 1
Effluent VFAs concentration with and without CO2 sequestration

| | | | | | Theoretical H₂ | | Total | | Theoretical / |
|---|---|---|---|---|---|---|---|---|---|
| | | | | | (L/d) | | Theoretical | Measured | Measured |
| | | Acetic | Propionic | Butyric | from | from | H₂ | H₂ | H₂ |
| | | g/L | | | acetic | butyric | L/d | L/d | % |
| OLR-1 | Without | 2.72 ± 0.19 | 1.00 ± 0.09 | 0.90 ± 0.09 | 48.2 | 13.6 | 61.9 | 53.0 | 117 |
| | KOH | | | | | | | | |
| | With | 3.92 ± 0.29 | ND | 0.48 ± 0.13 | 69.5 | 5.8 | 75.3 | 63.9 | 118 |
| | KOH | | | | | | | | |
| OLR-2 | Without | 5.77 ± 0.42 | 1.00 ± 0.07 | 0.78 ± 0.15 | 102.2 | 9.4 | 111.6 | 102.2 | 110 |
| | KOH | | | | | | | | |
| | With | 8.46 ± 0.46 | ND | 0.38 ± 0.10 | 149.9 | 4.6 | 154.5 | 130.0 | 119 |
| | KOH | | | | | | | | |

### 3.4 SRT and Biomass Yield

Table 2 shows the effluent and reactor VSS concentrations and values of the SRT and biomass yields. An increase in the effluent and reactor VSS was observed after sequestering CO₂ from the headspace, leading to an increase in the SRT from 2.5 to 2.67 d in case of OLR-1 and from 2.03 to 2.31 in case of OLR-2.

Biomass yields, calculated based on the converted SCOD, decreased after sequestering CO₂ from the headspace. For OLR-1 and OLR-2 the biomass yields decreased from 0.27 to 0.25 gVSS/gSCODconverted and from 0.22 to 0.21 gVSS/gSCODconverted, respectively.

TABLE 2
SRT and biomass calculations with and without CO₂ sequestration

| | | VSS | VSS | TS | TS | | TCOD | Biomass Yield |
|---|---|---|---|---|---|---|---|---|
| | | effluent | reactor | effluent | reactor | SRT | converted | gVSS/gTCOD |
| | | g/L | g/L | g/d | g | d | g/L | converted |
| OLR-1 | Without | 0.76 | 5.7 | 15.96 | 39.9 | 2.50 | 2.36 | 0.32 |
| | KOH | | | | | | | |
| | With | 0.80 | 6.4 | 16.80 | 44.8 | 2.67 | 2.76 | 0.29 |
| | KOH | | | | | | | |
| OLR-2 | Without | 1.20 | 7.3 | 25.20 | 51.1 | 2.03 | 4.32 | 0.28 |
| | KOH | | | | | | | |
| | With | 1.34 | 9.3 | 28.14 | 65.1 | 2.31 | 5.32 | 0.25 |
| | KOH | | | | | | | |

### 3.5 COD Mass Balance

Table 3 shows the COD mass balance data with a closure of 94±3% that verifies the reliability of the data. The COD balance was calculated considering input and output TCOD as well as equivalent COD for the produced H₂. An average COD reduction of 31±4% was achieved, which is consistent with Hafez et al. [2010] who observed 30% reduction in the COD.

TABLE 3
COD mass balance with and without CO₂ sequestration

| | | | | | | | | | COD |
|---|---|---|---|---|---|---|---|---|---|
| | | | | | | | | | mass |
| | | TCODin | | TCODout | | H₂ production | | | balance* |
| | | g/L | g/d | g/L | g/d | L/d | g/d | g COD/d | % |
| OLR-1 | Without | 9 | 180 | 6.2 | 135 | 53 | 4 | 34 | 94 |
| | KOH | | | | | | | | |
| | With | 9 | 180 | 5.8 | 123 | 63 | 5 | 40 | 90 |
| | KOH | | | | | | | | |
| OLR-2 | Without | 17 | 360 | 12.8 | 286 | 101 | 8 | 64 | 97 |
| | KOH | | | | | | | | |
| | With | 17 | 360 | 11.8 | 250 | 132 | 10 | 83 | 93 |
| | KOH | | | | | | | | |

*COD balance (%) = [H₂ produced (gCOD/d) + TCODout (g/d)] * 100/[TCODin (g/d)]

### 3.5 pH, Buffer and KOH Requirements

Reactor pH was maintained at 5.2±0.2 during the experiment using a buffer solution of 168 g/L NaHCO3. Buffer concentrations of 3 and 5 g NaHCO₃/L in the feed were kept constant before and after CO₂ sequestration from the headspace for both OLR-1 and OLR-2, respectively. It is noteworthy that using KOH in the headspace for CO₂ sequestration decreased the NaHCO₃ buffer consumption by the pH controller to only 16% of its consumption before adding the KOH, while overall NaHCO₃ buffer consumption i.e. feed and reactor pH control system decreased by 58%. Table 4 shows buffer concentrations used in the feed and consumed by the pH controller to maintain a constant pH of 5.2±0.2 during H₂ production.

Theoretical KOH consumption of 117 and 174 g/d for OLR-1 and OLR-2, respectively were calculated based on the experimental CO₂ production rates and a theoretical KOH consumption of 1.27 g KOH/g CO₂ (Equation 6). However, the experimental KOH consumption rates were observed to be 136 and 196 g/d for OLR-1 and OLR-2, respectively with an increase of 14% and 11% over the theoretical rates.

KOH+CO₂-->KHCO₃ (6)

Overall alkalinity consumption including both NaHCO₃ and KOH was calculated to be 120 and 195 mgCaCO₃/d before KOH application and 173 and 256 mgCaCO₃/d after KOH application for both OLR-1 and OLR-2, respectively. Although the overall alkalinity consumption increased by 44% and 31% in OLR-1 and OLR-2, respectively, this is outweighed by the increase in the H₂ production yields and rates and by obtaining 100% H₂. In addition, the KHCO₃ produced can be recycled and used as a buffer, which will reduce the overall buffer consumption.

TABLE 4
Buffer and KOH requirements

| | | | NaHCO₃ added | | | | | | |
|---|---|---|---|---|---|---|---|---|---|
| | | | | | | | | Total | |
| | | | | | pH controller | | | | g |
| | | | | | | Soln. | | | NaHCO₃/g |
| | | | Feed | | | conc. | | | glucose |
| | | pH | g/L | g/d | mL/d | g/L | g/d | g/d | feed |
| OLR-1 | Without | 5.2 ± 0.2 | 3 | 63 | 825 | 168 | 139 | 202 | 1.2 |
| | KOH | | | | | | | | |
| | With | 5.2 ± 0.2 | 3 | 63 | 140 | 168 | 24 | 87 | 0.52 |
| | KOH | | | | | | | | |
| OLR-2 | Without | 5.2 ± 0.2 | 5 | 105 | 1320 | 168 | 222 | 327 | 1.0 |
| | KOH | | | | | | | | |
| | With | 5.2 ± 0.2 | 5 | 105 | 190 | 168 | 32 | 137 | 0.41 |
| | KOH | | | | | | | | |

As is apparent from the exemplary embodiments of the process of this disclosure, continuous removal of CO₂ from the headspace shifted the H₂ producing pathways forward, increasing H₂ yields by 23% to 3.1 mol/mol and H₂ production rates by 23.5%. Sequestering CO₂ affected the rates of H₂ production as well as the ΔG of the thermodynamically unfavorable pathway that consumes propionate and produces H₂ and acetate. Effluent acetate concentration increased by 45% after applying KOH in the headspace, while butyrate concentration decreased to 51% of its value without sequestering CO₂. CO₂ sequestration changed the propionate consumption pathway to be thermodynamically favourable producing more acetate and H₂. Although buffer consumption for pH control after CO₂ sequestration was reduced to 42% of its original rate before CO₂ removal, overall alkalinity consumption considering the trap KOH exhausted, increased by 36% to 44%. In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art without departing from the scope, which is defined solely by the claims appended hereto.

### REFERENCES

1. Nuri Azbar, David Levin. State of the art and Progress in Production of Biohydrogen. Bentham Science Publishers, 2012
2. D. C. Dayton. Fuel Cell Integration—A Study of the Impacts of Gas Quality and Impurities. National Renewable Energy Laboratory, 2001
3. Larminie J, Dicks A. Fuel cell systems explained. New York: Wiley, 2000
4. Sompong O-Thong, Poonsuk Prasertsan, Nils-Kare Birkeland. Evaluation of methods for preparing hydrogen-producing seed inocula under thermophilic condition by process performance and microbial community analysis. Bioresource Technology 2009; 100: 909-918
5. David B. Levin, Lawrence Pitt, Murray Love. Biohydrogen production: prospects and limitations to practical application. International Journal of Hydrogen Energy 2004; 29: 173-185
6. Claire N. Sawyer, Perry L. McCarty, Gene F. Parkin. Chemistry for Environmental Engineering and Science (5^{th} edition). McGraw-Hill Companies, Inc. 2003
7. Kaushik Nath, Debabrata Das. Improvement of fermentative hydrogen production: various approaches. Appl Microbiol Biotechnol 2004; 65: 520-529
8. I. Hussy, F. R. Hawkes, R. Dinsdale, D. L. Hawkes. Continuous fermentative hydrogen production from sucrose and sugarbeet. International Journal of Hydrogen Energy 2005; 30: 471-483
9. Dong-Hoon Kim, Sun-Kee Han, Sang-Hyoun Kim, Hang-Sik Shin. Effect of gas sparging on continuous fermentative hydrogen production. International Journal of Hydrogen Energy 2006; 31: 2158-2169
10. Jeremy T. Kraemer, David M. Bagley. Improving the yield from fermentative hydrogen production. Biotechnol Lett 2007; 29: 685-695
11. Biswajit Mandal, Kaushik Nath. Improvement of biohydrogen production under decreased partial pressure of H₂ by Enterobacter cloacae. Biotechnol Lett 2006; 28: 831-835
12. Teh-Ming Liang, Sheng-Shung Cheng, Kung-Long Wu. Behavioural study on hydrogen fermentation reactor installed with silicone rubber membrane. International Journal of Hydrogen Energy 2002; 27: 1157-1165
13. Wooshin Park, Seung H. Hyun, Sang-Eun Oh, Bruce E. Logan, In S. Kim. Removal of headspace biological hydrogen production. Environ Sci Technol 2005; 39: 4416-4420
14. Bradley E. Jackson, Michael J. McInerney. Anaerobic microbial metabolism can proceed close to thermodynamic limits. Nature 2002; 415: 454-456
15. Hisham Hafez, George Nakhla, Hesham El Naggar. Biological hydrogen production from corn-syrup waste using a novel system. Energies 2009; 2: 445-455
16. APHA, AWWA, WEF. Standard methods for examination of water and wastewater. 19^{th} ed; 1995
17. Hisham Hafez, George Nakhla, M. Hesham El. Naggar, Elsayed Elbeshbishy, Bita Baghchehsaraee. Effect of organic loading on a novel hydrogen bioreactor. International Journal of Hydrogen Energy 2010; 35: 81-92
18. Zhen-Peng Zhang, Kuan-Yeow Show, Joo-Hwa Tay, David Tee Liang, Duu-Jong Lee, Wen-Ju Jiang. Effect of hydraulic retention time on biohydrogen production and anaerobic microbial community. Process Biochemistry 2006; 41: 2118-2123
19. Zhen-Peng Zhang, Kuan-Yeow Show, Joo-Hwa Tay, David Tee Liang, Duu-Jong Lee, Wen-Ju Jiang. Rapid formation of hydrogen-producing granules in an anaerobic continuous stirred tank reactor induced by acid incubation. Biotechnology and Bioengineering 2007; 96: 1040-1050
20. Kuan-Yeow Show, Zhen-Peng Zhang, Joo-Hwa Tay, David Tee Liang, Duu-Jong Lee, Wen-Ju Jiang. Production of hydrogen in a granular sludge-based anaerobic continuous stirred tank reactor. International Journal of Hydrogen Energy 2007; 32: 4744-4753
21. Shu-Yii Wu, Chun-Hsiung Hung, Chiu-Yue Lin, Ping-Jei Lin, Kuo-Shing Lee, Chi-Num Lin, Fang-Yuan Chang, Jo-Shu Chang. HRT-dependent hydrogen production and bacterial community structure of mixed anaerobic microflora in suspended, granular and immobilized sludge systems using glucose as the carbon substrate. International Journal of Hydrogen Energy 2008; 33: 1542-1549
22. Zhen-Peng Zhang, Kuan-Yeow Show, Joo-Hwa Tay, David Tee Liang, Duu-Jong Lee, Ay Su. The role of acid incubation in rapid immobilization of hydrogen-producing culture in anaerobic upflow column reactors. International Journal of Hydrogen Energy 2008; 33: 5151-5160
23. Kuan-Yeow Show, Zhen-Peng Zhang, Joo-Hwa Tay, David Tee Liang, Duu-Jong Lee, Nanqi Ren, Aijie Wang. Critical assessment of anaerobic processes for continuous biohydrogen production from organic wastewater. International Journal of Hydrogen Energy 2010; 35: 13350-13355
24. F. R. Hawkes, R. Dinsdale, D. L. Hawkes, I. Hussy. Sustainable fermentative hydrogen production: challenges for process optimisation. International Journal of Hydrogen Energy 2002; 27: 1339-1347
V. A. Vavilin, S. V. Rytow, L. Ya Lokshina. Modelling hydrogen partial pressure change as a result of competition between the butyric and propionic groups of acidogenic bacteria. Bioresource Technology 1995; 54: 171-177

What is claimed is:

1. A method for producing hydrogen by dark fermentation from organic material, comprising the steps of
introducing organic material and microorganisms into a completely mixed bioreactor for breaking down the organic material into products including H₂ gas, CO₂ gas, volatile fatty acids, and alcohols by dark fermentation;
continuously sequestering CO₂ gas within a headspace of the bioreactor for capturing the CO₂ as bicarbonate within the headspace; and
recovering at least a portion of the H₂ gas from the headspace under vacuum, whereby the recovered H₂ gas is substantially free of CO₂.

2. The method of claim 2, wherein the H₂ gas is continuously recovered from the headspace.

3. The method of claim 2, wherein the step of continuously sequestering CO₂ within the headspace includes the further step of discontinuously removing at least a portion of the bicarbonate from the headspace.

4. The method of claim 1, wherein the step of continuously capturing CO₂ includes continuously maintaining a metal hydroxide in the headspace for binding of the gaseous CO₂ as metal bicarbonate within the headspace.

5. The method of claim 5, wherein the metal hydroxide is used in solid form.

6. The method of claim 6, wherein the metal hydroxide is an alkali metal hydroxide.

7. The method of claim 7, wherein the metal hydroxide is KOH or NaOH.

8. The method of claim 8, wherein the metal hydroxide is in the form of 100% pure KOH or NaOH pellets.

9. The method of claim 1, comprising the further step of maintaining a concentration of microorganisms in the completely mixed bioreactor at a preselected value.

10. The method of claim 10, comprising the further step of controlling the pH of the completely mixed bioreactor.

11. The method of claim 11, wherein the pH of the completely mixed bioreactor is maintained within a range of 3 to 6.8.

12. The method of claim 12, wherein the pH is maintained at about 5.2.

13. A system for producing hydrogen, methane, volatile fatty acids, and alcohols from organic material, comprising:
a completely mixed bioreactor for dark fermentation;
an input for supplying to the bioreactor microorganisms and the organic material to be broken down into products including H₂ gas, CO₂ gas, volatile fatty acids (VFA) and alcohols;
a CO₂ trap in a headspace of the reactor, the CO₂ trap including a solid hydroxide for sequestration of the CO₂ gas from the headspace and capture of the CO₂ as bicarbonate within the headspace;
a gas output for removal of a gas effluent including H₂ gas from the headspace; and
a liquid output for removing a first liquid effluent including at least a portion of the microorganisms, the volatile fatty acids, and the alcohols from the bioreactor.

14. The system of claim 14, wherein the completely mixed bioreactor is a reactor selected from the group consisting of a single continuously stirred tank reactor, a multi-stage continuously stirred tank reactor, an up-flow anaerobic sludge blanket reactor, an expanded bed granular sludge blanket reactor, a down-flow anaerobic granular media reactor, an up-flow anaerobic granular media reactor, an anaerobic baffled tank reactor, an anaerobic migrating blanket reactor, and an anaerobic fluidized bed bioreactor.

15. The system of claim 14, wherein the trap includes a solid metal hydroxide.

16. The system of claim 16, wherein the trap includes a solid alkali metal hydroxide.

17. The system of claim 17, wherein the trap includes KOH, or NaOH.

18. The system of claim 18, wherein the KOH is in the form of pellets of 100% KOH, or NaOH.

19. The system of claim 14, comprising two or more CO₂ traps separately removable from the headspace for removal of the bicarbonate during continuous operation of the reactor.

20. The system of claim 14, further comprising a gravity settler in fluid communication with the liquid output for separating the first liquid effluent into a settled out first biomass including at least a portion of the microorganisms and a second liquid effluent including at least a portion of the volatile fatty acids, the alcohols and the microorganisms; and means for feeding the first biomass from the gravity settler to the completely mixed bioreactor for maintaining a concentration of microorganisms in the completely mixed bioreactor at a preselected value.

21. The system of claim 21, further comprising a dispenser for dispensing chemicals for pH adjustment into the completely mixed bioreactor.

22. The system of claim 22, further comprising a temperature controller for controlling a temperature of the bioreactor.
